(19) United States
(12) Patent Application Publication
(10) Pub. No.: US 2026/0216521 A1
(43) Pub. Date: Jul. 30, 2026

(54) Systems And Methods With Backup Communication Links

(71) Applicant: Cochlear Limited, Macquarie University, NSW (AU)

(72) Inventors: Helmut Christian Eder, Sydney (AU); Rowan Humphreys, Sydney (AU)

(73) Assignee: Cochlear Limited, Macquarie University, NSW (AU)

(21) Appl. No.: 19/139,825

(22) PCT Filed: Dec. 1, 2023
(86) PCT No.: PCT/IB2023/062143

§ 371 Date: Jun. 16, 2025

Related U.S. Application Data
(60) Provisional application No. 63/434,859, filed on Dec. 22, 2022.

(51) Int. Cl. A61N 1/372; A61N 1/05; A61N 1/08; A61N 1/36; H04W 76/15; H04W 76/18; H04W 76/19

(52) U.S. Cl. CPC A61N 1/37276; A61N 1/0541; A61N 1/08; A61N 1/36038; A61N 1/37217; H04W 76/15; H04W 76/18; H04W 76/19

(57) ABSTRACT

An implant system includes an implantable component and an external component. The implant system switches communication between the implantable component and the external component from a first communication link to a second communication link in response to detecting interference in the first communication link above a threshold. The implant system switches communication between the implantable and external components from the second communication link back to the first communication link in response to detecting interference in the first communication link below the threshold.

### CROSS REFERENCE TO RELATED APPLICATION

This patent application claims priority to U.S. provisional patent application 63/434,859, filed Dec. 22, 2022, which is incorporated by reference herein in its entirety.

### FIELD OF THE DISCLOSURE

The present disclosure relates to systems and methods that transmit signals between components through backup links.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### BRIEF SUMMARY

According to a first aspect of the present invention, a medical device system comprises a first component, wherein the medical device system switches communication between the first component and a second component of the medical device system from a first communication link to a second communication link in response to an error in the first communication link.

According to a second aspect of the present invention, an implant system comprises an external component, wherein the implant system switches communication between the external component and an implantable component of the implant system from a first communication link to a second communication link in response to interference in the first communication link.

According to a third aspect of the present invention, a method comprises communicating between external and implantable components of an implant system through a first signal transmission link; and communicating between the external and the implantable components through a second signal transmission link in response to detecting interference in the first signal transmission link.

According to a fourth aspect of the present invention, a non-transitory computer readable storage medium comprises instructions stored thereon that, when executed by a computing system, cause the computing system to detect when an error rate of bits transmitted through a first link between external and implantable components of an implant system reaches a threshold; and cause signals to be transmitted through a second link between the external and the implantable components in response to the error rate reaching the threshold.

According to a fifth aspect of the present invention, an implant system includes an external component comprising first and second antennae and first and second transceivers, and an implantable component comprising third and fourth antennae and third and fourth transceivers. The implant system transmits first signals between the implantable component and the external component through the first transceiver, the first antenna, the third antenna, and the third transceiver in response to interference in the first signals being less than a threshold. The implant system transmits second signals between the implantable component and the external component through the second transceiver, the second antenna, the fourth antenna, and the fourth transceiver in response to the interference in the first signals being greater than the threshold.

### BRIEF DESCRIPTION OF THE DRAWINGS

FIG. 1 is a diagram of an example of an auditory prosthesis that can include one or more embodiments disclosed herein.

FIG. 2 is a diagram that illustrates details of an example of the auditory prosthesis of FIG. 1 having a main link and a backup link used for transmitting signals between the external and implantable components of the auditory prosthesis.

FIG. 3A is a flow chart that illustrates examples of operations that can be performed to manage communications between an external component and an implantable component of an implant system using a main link and a backup link.

FIG. 3B is a flow chart that illustrates examples of operations that can be performed as alternatives to performing a subset of the operations of FIG. 3A.

FIG. 4 is a diagram that illustrates an example of a suitable computing system that can perform any of the operations or functions disclosed herein.

### DETAILED DESCRIPTION

Merely for ease of description, the techniques presented herein are primarily described herein with reference to an illustrative medical device, namely a cochlear implant system. However, it is to be appreciated that the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant system, corresponds to a disclosure of another embodiment of using such teaching with another hearing prostheses, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. The techniques presented herein can also be implemented in dedicated tinnitus therapy devices and tinnitus therapy device systems.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to evoking a hearing percept corresponds to a disclosure of evoking other types of neural percepts in other embodiments, such as a visual/sight percept, a tactile percept, a smell precept or a taste percept, unless otherwise indicated and/or unless the art does not enable such. Any disclosure herein of a device, system and/or method that is used to or results in ultimate stimulation of the auditory nerve corresponds to a disclosure of an analogous stimulation of the optic nerve utilizing analogous components, methods, and systems.

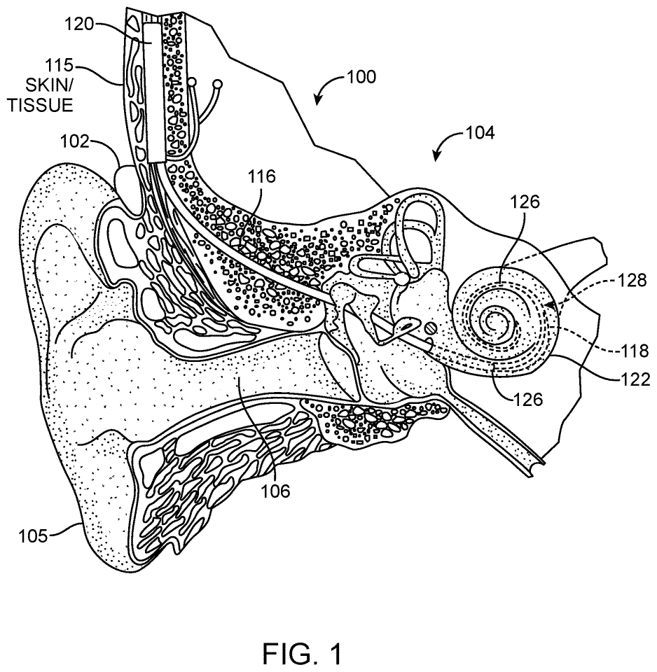

FIG. 1 is a diagram illustrating an example of an auditory prosthesis 100 that can include one or more embodiments disclosed herein. The auditory prosthesis 100 of FIG. 1 is an example of a cochlear implant system (e.g., a mostly implantable cochlear implant system or MICI) that includes an external component 102 and an internal/implantable component 104. The external component 102 is positioned by an auricle 105 of the recipient and is configured to be attached to, and worn adjacent to, the recipient's ear. However, the external component 102 can have other arrangements, such as an off the ear (OTE) processing unit (e.g., a component configured to be magnetically coupled to the recipient's head), an in-the-canal unit that is configured to be located in the recipient's ear canal 106, etc.

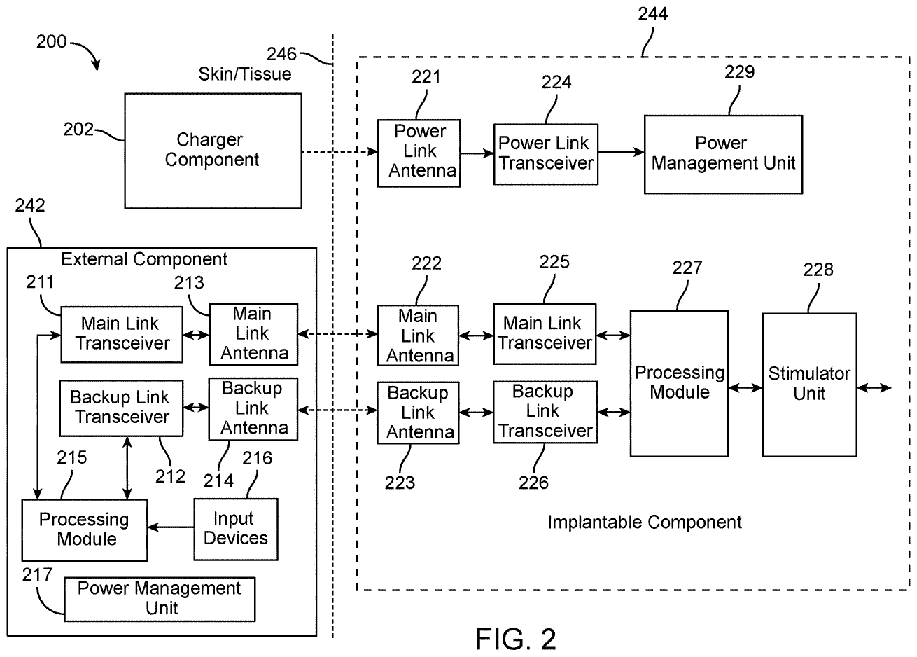

The implantable component 104 comprises an implant body 120, a lead region 116, and an elongated intra-cochlear stimulating assembly 118, all configured to be implanted under the skin/tissue (tissue) 115 of the recipient. The implant body 120 comprises a hermetically sealed housing that houses various components, examples of which are disclosed herein in further detail with respect to FIG. 2. The housing of implant body 120 operates as a protective barrier between the components within the housing of implant body 120 and the recipient's tissue and bodily fluid.

Stimulating assembly 118 is configured to be at least partially implanted in the recipient's cochlea 122. Stimulating assembly 118 includes a plurality of longitudinally spaced intra-cochlear electrical stimulating contacts (electrodes) 126 that collectively form a contact or electrode array 128 for delivery of electrical stimulation (current) to the recipient's cochlea 122. Stimulating assembly 118 extends through an opening in the recipient's cochlea (e.g., cochleostomy, the round window, etc.) and has a proximal end connected to a stimulator unit in implant body 120 via lead region 116 and a hermetic feedthrough (not shown in FIG. 1). Lead region 116 includes a plurality of conductors (wires) that electrically couple the electrodes 126 to the stimulator unit.

It may be desirable for an implant system, such as auditory prosthesis 100, to use a low power link to transmit signals between external and implantable components of the implant system in order to reduce power consumption of the implant system and to extend battery life. A magnetic induction link is typically a very low power link that can be used by an implant system for the wireless transmission of signals, such as audio signals indicating audio data. As an example, the auditory prosthesis 100 of FIG. 1 can use a magnetic induction link to transmit audio signals from the external component 102 to the implantable component 104.

Although magnetic induction links are typically very low power links, magnetic induction links have some drawbacks that may make these links more susceptible to interference (i.e., electromagnetic interference). For example, the receivers in magnetic induction links are designed to be extremely sensitive in order to detect the low power signals from the transmitters. In addition, the transmit and receive antennae in magnetic induction links are aligned as closely as possible in order to ensure signal quality. Because of the characteristics of the magnetic field used to transmit data, magnetic induction links tend to be close range links. Also, the frequencies used for transmitting data in magnetic induction links tend to be the same, or similar, to the frequencies that are used by many other commercial applications, such as anti-theft scanners, short-wave radio communications, airplane communications, etc. All of these factors can cause magnetic induction links to be more susceptible to interference than is desirable for many types of implant systems.

External interference can interrupt the transmission of signals in a magnetic induction link in an implant system, potentially interrupting the normal operation of the implant system and negatively impacting the experience of the recipient regarding use of the implant system. Recipients of cochlear implant systems typically rely on the cochlear implant systems to provide continuous audio communication under any circumstances. Therefore, it is not considered to be acceptable for a cochlear implant system to experience audio drop-outs between the external and implantable components that are caused by interference, even if the audio drop-outs occur infrequently. The occurrence of audio-drop outs in a cochlear implant system can seriously undermine a recipient's expectations regarding the operation and reliability of the cochlear implant system.

According to some embodiments disclosed herein, an implant system includes a main link that is used as the primary means of communicating signals between an external component and an implantable component of the implant system. The main link can be, for example, a low power link, such as a magnetic induction link, that the implant system prioritizes for signal transmission between the external and implantable components to reduce power consumption. The implant system also includes a backup link that is used as a secondary means of communicating signals between the external and implantable components of the implant system. The implant system monitors interference (e.g., an error rate) in the communication through the main link, for example, using a processor. If the implant system determines that the interference in the main link reaches or exceeds a threshold (e.g., that is indicative of the possibility of audio drop-outs), the implant system switches communication between the implantable and external components from the main link to the backup link. The backup link can provide continuous (e.g., audio drop-out free) communication between the external and implantable components, while the main link is experiencing interference. The implant system switches communication between the implantable and external components back to the main link in response to the interference in the main link decreasing below the threshold. The implant system can be any type of implant system, including, for example, any type of cochlear implant system, bone conduction device, middle ear auditory prosthesis, direct acoustic stimulator, auditory brain stimulator, retinal prosthesis, or any other type of prothesis. Further details of exemplary embodiments are disclosed herein below.

FIG. 2 is a diagram that illustrates details of an example of a medical device system 200 having a main link and a backup link used for communicating signals between the external and implantable components. Medical device system 200 can be any type of medical device or implant system, such as an auditory prothesis (e.g., auditory prosthesis 100 of FIG. 1), a retinal prosthesis, a vestibular device, a seizure device, a sleep apnea device, a tinnitus therapy device, a pacemaker, a drug delivery system, a defibrillator, a functional electrical stimulation device, a catheter, an electroporation device, etc. As shown in FIG. 2, the medical device system 200 includes an external component 242, an implantable component 244, and a charger component 202. The implantable component 244 can include a power link antenna 221, a power link transceiver 224, and a power management unit 229 that can, for example, be located in the implant body of a prothesis (such as implant body 120 in the auditory prothesis 100). Charger component 202 can be applied externally to the recipient to provide power through a short range power link to the implantable component 244. For example, the charger component 202 can be applied on the skin/tissue 246 of the recipient (e.g., above the implant body 120) to transfer power to the power management unit 229 through the power link antenna 221 and the power link transceiver 224 (e.g., by capacitive and/or inductive power transfer). The power management unit 229 can, for example, include a battery that is charged by power received from the charger component 202 through the power link antenna 221 and the power link transceiver 224.

In the example of FIG. 2, the implantable component 244 also includes a main link antenna 222, a backup link antenna 223, a main link transceiver 225, a backup link transceiver 226, a processing module 227, and a stimulator unit 228, each of which can, for example, be within the implant body of a prothesis, such as implant body 120. The power management unit 229 can provide power to each of the electrical components in the implantable component 244, including antennae 221-223, transceivers 224-226, processing module 227, and stimulator unit 228. In embodiments in which medical device system 200 is auditory prothesis 100, the implantable component 244 is implantable component 104, and the external component 242 is the external component 102. In these embodiments, the implantable component 104/244 also includes the lead region 116 and stimulating assembly 118 that includes the electrical stimulating contacts (electrodes) 126 that collectively form contact array 128, and the lead region 116 is coupled to the stimulator unit 228.

As shown in FIG. 2, the external component 242 includes a main link transceiver 211, a backup link transceiver 212, a main link antenna 213, a backup link antenna 214, a processing module 215, one or more input devices 216, and a power management unit 217. The power management unit 217 can provide power to each of the electrical components in the external component 242, including main link transceiver 211, backup link transceiver 212, main link antenna 213, backup link antenna 214, processing module 215, and one or more input devices 216. The one or more input devices 216 can include sound input devices (e.g., microphones positioned by auricle 105 of the recipient, telecoils, etc.), one or more auxiliary input devices (e.g., audio ports, such as a Direct Audio Input (DAI), data ports, such as a Universal Serial Bus (USB) port, cable port, etc.), and/or a wireless transmitter/receiver (transceiver) configured to capture/receive input signals from outside medical device system 200.

The main link antennae 213 and 222, the main link transceivers 211 and 225, the backup link antennae 214 and 223, the backup link transceivers 212 and 226, input devices 216, and the processing modules 215 and 217 can be provided in any type of medical device, such as an auditory prothesis, a retinal prosthesis, a sensory prothesis, a vestibular device, a seizure device, a sleep apnea device, a tinnitus therapy device, a pacemaker, a drug delivery system, a defibrillator, a functional electrical stimulation device, a catheter, an electroporation device, etc. Each of the processing module 215 in the external component 242 and the processing module 227 in the implantable component 244 can perform one or more processing functions for any type of medical device. In an auditory prothesis, such as the auditory prosthesis 100, the processing modules 215 and 217 can include a number of elements, such as an environmental classifier, a sound processor, and/or an individualized own voice detector. Each of the environmental classifier, the sound processor, and the individualized own voice detector in one or both of the processing modules 215 and/or 227 can be implemented by one or more processor circuit devices (e.g., one or more Digital Signal Processors (DSPs), one or more processing cores, one or more processing integrated circuits, etc.), firmware, software, etc. arranged to perform operations described herein. That is, the environmental classifier, the sound processor, and the individualized own voice detector can each be implemented as firmware elements, partially or fully implemented with digital logic gates in one or more application-specific integrated circuits (ASICs), partially or fully in software, etc.

The medical device system 200 of FIG. 2 includes a main link and a backup link for communicating signals between external component 242 and implantable component 244. The signals transmitted through the main and backup links can, for example, indicate audio data, stimulation data for stimulating an auditory nerve of the recipient (also referred to herein as auditory stimulation data), other types of data, control codes, control information, software visual data for visual devices (e.g., for a retinal prosthesis), tactile data for a tactile prosthesis, olfactory data for a smell prosthesis, taste data for a taste prosthesis, other types of sensory data, electrical stimulation data for other types of electrical stimulation devices (such as pacemakers, defibrillators, seizure devices, sleep apnea devices, electroporation devices, etc.). The medical device system 200 uses the main link as the primary link for communicating signals between the external component 242 and the implantable component 244. The main link can be, for example, a wireless, low power communication link that the medical device system 200 prioritizes for signal transmission between the external and implantable components to reduce power consumption in both the external and implantable components.

The main link includes an antenna and a transceiver in each of the external and implantable components, which are shown in FIG. 2 as the main link transceiver 211, the main link antenna 213, the main link antenna 222, and the main link transceiver 225. As a specific example that is not intended to be limiting, the main link can include a wireless near-field magnetic induction communication system that transmits communications through low power magnetic fields between antennae 213 and 222. According to this example, each of the antennae 213 and 222 can include one or more magnetic induction coils that modulate and demodulate information in a carrier signal transmitted using magnetic fields.

The backup link in the medical device system 200 also includes an antenna and a transceiver in each of the external and implantable components, which are shown in FIG. 2 as the backup link transceiver 212, the backup link antenna 214, the backup link antenna 223, and the backup link transceiver 226. The medical device system 200 uses the backup link as a secondary link for communicating signals between the external component 242 and the implantable component 244, if interference in the main link reaches or exceeds a threshold that indicates that the main link may experience, for example, audio drop-outs. As an example that is not intended to be limiting, the backup link can be a radio frequency link that transmits radio frequency (RF) signals (RF electromagnetic waves) between antennae 214 and 223. The backup link can transmit and receive signals at any frequencies, as long as interference in one of the links does not cause interference in the other link. The frequency bands of the main and backup links should preferably be appropriately spaced apart to avoid interference in one of the links from causing interference in the other link. As specific examples, the backup link can transmit and receive radio frequency carrier signals in the 400 megahertz (MHz), 900 MHz, or 2.4 gigahertz (GHz) bands.

Further details of exemplary communications between the external component 242 and the implantable component 244 of the medical device system 200 are now described. The input devices 216 can receive input signals (e.g., audio input signals) from one or more external sources and provide the input signals to the processing module 215 in the external component 242. The processing module 215 is configured to process the input signals received from input devices 216 to generate output signals. For example, the processing module 215 (e.g., one or more processing elements implementing firmware, software, etc.) can be configured to perform one or more sound processing functions (e.g., using an environmental classifier, a sound processor, and/or an individualized own voice detector) on input audio signals received from input devices 216 to generate output audio signals. In the embodiment of FIG. 2, the processing module 215 provides the output signals to the processing module 227 in the implantable component 244 (e.g., in an encoded manner) through one or both of the main link and/or the backup link, which are described in further detail below.

The processing module 227 receives the output signals (e.g., the output audio signals) generated by the processing module 215 and transmitted through one or both of the main link and/or the backup link. The processing module 227 can perform one or more processing functions (e.g., sound processing functions using an environmental classifier, a sound processor, and/or an individualized own voice detector) on the output signals received from the processing module 215 to generate stimulation control signals for use in stimulating a recipient. Stated differently, the processing module 227 (e.g., one or more processing elements implementing firmware, software, etc.) is configured to convert the output signals of the processing module 215 into stimulation control signals that represent electrical stimulation for delivery to the recipient (e.g., the recipient's cochlea in an auditory prothesis).

The processing module 227 provides the stimulation control signals to the stimulator unit 228. The stimulator unit 228 is configured to utilize the stimulation control signals to generate output stimulation signals (e.g., current signals) for delivery to the recipient. As an example, the stimulator unit 228 can use the stimulation control signals to generate electrical stimulation signals for stimulating one or more of the stimulating contacts 126 in contact array 128 in auditory prothesis 100. In this way, auditory prosthesis 100 electrically stimulates the recipient's auditory nerve cells, bypassing absent or defective hair cells that normally transduce acoustic vibrations into neural activity, in a manner that causes the recipient to perceive one or more components of the input audio signals.

In some embodiments, the stimulator unit 228 can be configured to receive signals from the recipient and to provide the signals received from the recipient to processing module 227. For example, in auditory prothesis 100, the stimulator unit 228 can be configured to receive signals from one or more of the stimulating contacts 126 in contact array 128 (e.g., in response to signals from the auditory nerve or from one or more of the other stimulating contacts 126) and to provide the signals received from one or more of the stimulating contacts 126 to processing module 227. The processing module 227 can perform one or more processing functions on the signals received from the stimulator unit 228 to generate output signals that are provided through one or both of the main link and/or the backup link to the processing module 215 in the external component 242 for one or more additional processing functions. Thus, the main link and the backup link are bi-directional links, as shown by the two-way arrows connecting the processing modules 215 and 227 via the main and backup links. Although in other embodiments, the main and backup links can be unidirectional links. Although some embodiments are disclosed herein in the context of medical device system 200, a main link and a backup link as shown in FIG. 2 can be used in any type of implant system including, for example, any type of cochlear implant system, bone conduction device, middle ear auditory prosthesis, direct acoustic stimulator, auditory brain stimulator, retinal prosthesis, or any other type of prothesis.

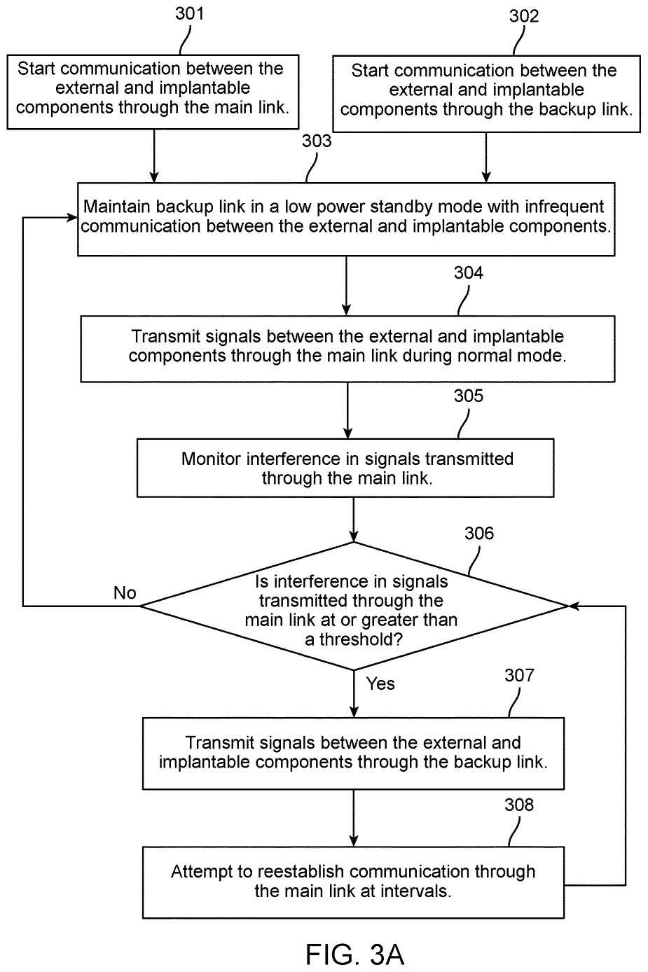

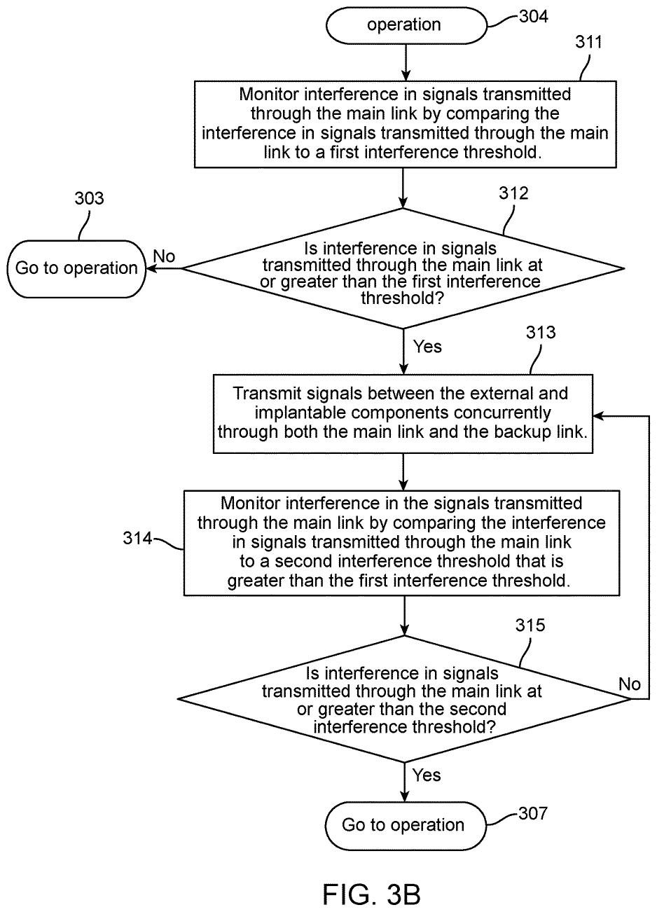

FIG. 3A is a flow chart that illustrates examples of operations that can be performed to manage communications between an external component and an implantable component of an implant system using a main link and a backup link. The operations of FIGS. 3A-3B are disclosed primarily in the context of the medical device system 200 of FIG. 2 as an example. Although, it should be understood that the operations of FIGS. 3A-3B can be performed for any type of medical device or implant system, including, for example, any type of cochlear implant system, bone conduction device, middle ear auditory prosthesis, direct acoustic stimulator, auditory brain stimulator, retinal prosthesis, sensory prothesis, or any other type of prothesis.

In operation 301, the medical device system starts communication between the external component and the implantable component through the main link. For example, the medical device system 200 can start communication between external component 242 and implantable component 244 in operation 301 through the main link components, including through main link transceiver 211, main link antenna 213, main link antenna 222, and main link transceiver 225. In operation 302, the medical device system starts communication between the external component and the implantable component through the backup link. For example, the medical device system 200 can start communication between external component 242 and implantable component 244 in operation 302 through the backup link components, including through backup link transceiver 212, backup link antenna 214, backup link antenna 223, and backup link transceiver 226. As an example, each of operations 301 and 302 can include an automated handshaking process for establishing communications through the respective link, before full communications begin through the respective link. The handshaking process can include, for example, exchanging signals indicative of communication protocols or parameters between the transceivers and the antennae in the external and implantable components in each of the main link and the backup link. Operations 301 and 302 can be performed in parallel (e.g., concurrently) or in series.

In operation 303, the medical device system maintains the backup link in a low power standby mode of operation (also referred to as a low power standby mode) with infrequent communication between the external and implantable components. The medical device system generates the infrequent communication through the backup link between the external and implantable components during the low power standby mode in operation 303 to ensure that full communication (e.g., continuous communication) can be subsequently started through the backup link in a short time. The infrequent communication through the backup link can, for example, include repeating one or more functions in the automated handshaking process described herein with respect to operation 302, for example, using backup link transceiver 212, backup link antenna 214, backup link antenna 223, and/or backup link transceiver 226. The infrequent communication preferably causes the backup link to consume very little power (or no power) during the low power standby mode, so as not to negate the benefit of using a low power main link as the primary means of signal transmission during a normal mode of operation. The infrequent communication can involve, for example, transmitting signals through the backup link in intervals that are spaced apart by enough time (such as hundreds of milliseconds or one or more seconds) to cause the backup link to consume very little power in the low power standby mode.

Maintaining the backup link in the low power standby mode in operation 303 ensures that the backup link can stay established during normal mode without substantial interference. As an example, the backup link may experience interference if the backup link is near another device that transmits wireless signals through the same frequency band. If, for example, the backup link experiences interference (e.g., an error rate) that is greater than a threshold level, the medical device system can cause the infrequent communication through the backup link during the low power standby mode to switch to a different frequency band (e.g., a different channel). As a more specific example that is not intended to be limiting, the medical device system can switch the infrequent communication through the backup link during the low power standby mode in response to interference to a different channel using frequency-hopping spread spectrum (FHSS). The medical device system can use FFHS to rapidly change the carrier frequency used by the backup link between center frequencies of different sub-bands within an available frequency band.

In operation 304, the medical device system transmits signals between the external and implantable components through the main link during the normal mode of operation. For example, the medical device system 200 can transmit signals (e.g., signals indicating audio data, auditory stimulation data, software, control codes, visual data for a retinal prosthesis, tactile data for a tactile prosthesis, olfactory data for a smell prosthesis, taste data for a taste prosthesis, sensory data for other types of sensory prostheses, electrical stimulation data for other types of electrical stimulation devices (such as pacemakers, defibrillators, seizure devices, sleep apnea devices, electroporation devices), other types of data or codes, etc.) between the processing module 215 in the external component 242 and the processing module 227 in implantable component 244 in operation 304 through the main link components, including through main link transceiver 211, main link antenna 213, main link antenna 222, and main link transceiver 225. The stimulation data can be provided to the implantable component in stimulation control signals. As discussed above, the main link of FIG. 2 is a bidirectional communication link. Thus, the main link can transmit signals (e.g., indicating audio data, auditory stimulation data, etc.) from processing module 215 to processing module 227 for use by the stimulator unit 228 in stimulating the recipient (e.g., stimulating contacts 126). The main link can also transmit signals (e.g., indicating processed data) from processing module 227 to processing module 215.

In operation 305, the medical device system monitors interference in signals transmitted through the main link. As an example, medical device system 200 can monitor interference in signals transmitted through the main link using one or both of the processing module 215 and/or the processing module 227. The medical device system can, for example, monitor interference in signals transmitted through the main link by monitoring an error rate of bits transmitted through the main link in operation 305 using error detection and/or error correction techniques.

The medical device system can compare the interference in signals transmitted through the main link to an interference threshold in operation 305. As an example, the medical device system 200 can compare the interference in signals transmitted through the main link to the interference threshold using one or both of the processing module 215 and/or the processing module 227. In operation 306, the medical device system determines if the interference in signals transmitted through the main link is at or greater than the interference threshold. The medical device system can, for example, compare the error rate of bits transmitted through the main link to an error rate threshold and determine if the monitored error rate is at or greater than the error rate threshold in operation 306.

If the medical device system determines that the interference in signals transmitted through the main link is not at or greater than the interference threshold in operation 306 (i.e., the interference is lower than the interference threshold), the medical device system continues to maintain the backup link in the low power standby mode in operation 303. The medical device system also continues to transmit signals between the external and implantable components through the main link in operation 304. In addition, the medical device system continues to monitor interference in signals transmitted through the main link and compares the monitored interference to the interference threshold in operation 305.

If the medical device system determines that the interference in signals transmitted through the main link is at or greater than the interference threshold in operation 306, the medical device system switches communications from the main link to the backup link. The medical device system then transmits signals between the external and implantable components through the backup link in operation 307. The signals transmitted through the backup link in operation 307 can, for example, indicate audio data, auditory stimulation data, visual data, tactile data, olfactory data, taste data, sensory data, electrical stimulation data for various types of electrical stimulation devices (such as pacemakers, defibrillators, seizure devices, sleep apnea devices, electroporation devices), other types of data, control codes, software code, etc. The medical device system can switch communications from the main link to the backup link to provide continuous transmission of signals (e.g., signals indicating audio data, auditory stimulation data, control codes, software, visual data, tactile data, olfactory data, taste data, sensory data, electrical stimulation data for various types of electrical stimulation devices (such as pacemakers, defibrillators, seizure devices, sleep apnea devices, electroporation devices), other types of data, etc.) between the external and implantable components, with minimal or no loss of signal transmission (e.g., without causing audio drop-outs in auditory prothesis 100).

As an example, the medical device system 200 can transmit signals (e.g., signals indicating audio data, auditory stimulation data, software, other types of sensory data, control codes, etc.) between the processing module 215 in external component 242 and the processing module 227 in implantable component 244 in operation 307 through the backup link components, including through backup link transceiver 212, backup link antenna 214, backup link antenna 223, and backup link transceiver 226. As discussed above, the backup link can be a bidirectional link. Thus, the backup link can transmit signals from the processing module 215 to the processing module 227 for use by the stimulator unit 228 in stimulating the recipient in operation 307. The backup link can also transmit signals (e.g., indicating processed sensory data) from the processing module 227 to the processing module 215 in operation 307. If interference occurs in the current channel used by the backup link, the backup link can switch from the current channel to a different channel within an available frequency band using FHSS in response to detecting the interference in the current channel.

In some embodiments, the backup link uses a greater amount of power in the medical device system (e.g., from power management units 217 and 229) than the main link. For example, the backup link can transmit data at a higher bit rate than the main link. Therefore, while the medical device system is transmitting communications through the higher power backup link, the medical device system attempts to reestablish communication through the lower power main link at intervals (e.g., in the background) in operation 308. As examples, the medical device system 200 can attempt to reestablish communication through the main link using one or more of the processing module 215, the main link transceiver 211, the main link transceiver 225, and/or the processing module 227. The medical device system can, for example, attempt to reestablish communication through the lower power main link without imposing an unreasonable power draw on the medical device system (e.g., from power management units 217 and 229). As an example, the medical device system can attempt to reestablish communication through the main link in intervals spaced apart in time, rather than continuously. The intervals can, for example, be infrequent regular (or irregular) time intervals (e.g., spaced apart by 1-100 seconds).

The medical device system can, for example, attempt to reestablish communication through the main link by comparing interference in signals transmitted through the main link at intervals to the interference threshold in operation 308, as discussed above with respect to operation 305. If the medical device system determines that the interference in signals transmitted through the main link is at or greater than the interference threshold in operation 306, the medical device system continues to transmit the signals between the external and implantable components through the backup link in operation 307.

If the medical device system determines that the interference in signals transmitted through the main link at intervals is not at or greater than the interference threshold in operation 306 (e.g., less than the interference threshold), the medical device system switches communications between the external and implantable components from the backup link to the main link. The medical device system then returns the backup link to the low power standby mode in operation 303, and the medical device system transmits signals (indicating, e.g., audio data, auditory stimulation data, visual data, tactile data, olfactory data, taste data, sensory data, electrical stimulation data for various types of electrical stimulation devices (such as pacemakers, defibrillators, seizure devices, sleep apnea devices, electroporation devices), other types of data, software, control codes, etc.) between the external and implantable components through the main link again during normal mode in operation 304. Operations 305 and 306 are also performed again to monitor interference in the main link. According to another embodiment, the medical device system switches communications between the external and implantable components from the backup link to the main link only when the interference in signals transmitted through the main link has continuously remained below the interference threshold for a predetermined amount of time, rather than switching communications back to the main link immediately after the interference in the main link has decreased below the interference threshold.

Thus, the medical device system switches communications between the external and implantable components from the backup link back to the main link in response to successful signal transmission being reestablished through the main link (i.e., interference in the main link has decreased or resolved). The medical device system can switch communications from the backup link back to the main link to provide continuous transmission of signals (e.g., signals indicating audio data, auditory stimulation data, visual data, other types of sensory data, other types of data, software, control codes, etc.) between the external and implantable components, with minimal or no loss of signal transmission. For example, the medical device system can switch communications back to the main link without experiencing audio drop-outs.

According to an alternative embodiment, the medical device system can transmit signals (e.g., signals indicating audio data, auditory stimulation data, visual data, tactile data, olfactory data, taste data, sensory data, electrical stimulation data for various types of electrical stimulation devices (such as pacemakers, defibrillators, seizure devices, sleep apnea devices, electroporation devices), other types of data, software, control codes, etc.) between the external and implantable components concurrently through both the main link and the backup link if the medical device system determines that the interference in signals transmitted through the main link is at or greater than a first interference threshold. FIG. 3B is a flow chart that illustrates examples of operations that can be performed as alternatives to performing operations 305-306 in FIG. 3A. In the embodiment of FIG. 3B, operations 301-304 and 307-308 are performed as disclosed herein with respect to FIG. 3A, and operations 311-315 are performed instead of operations 305-306. After operation 304, the medical device system monitors interference in signals transmitted through the main link by comparing the interference in signals transmitted through the main link to a first interference threshold in operation 311. The first interference threshold can, for example, be equal to a bit error rate indicative of errors in audio data or auditory stimulation data transmitted through the main link that do not yet cause audio drop-outs in the main link. The medical device system can, for example, use forward error correction (FEC) to correct errors in signals transmitted through the main link in operation 311 when the error rate of signals transmitted through the main link is less than a second interference threshold that is greater than the first interference threshold.

In operation 312, the medical device system determines if the interference in signals transmitted through the main link is at or greater than the first interference threshold. If the medical device system determines that the interference in signals transmitted through the main link is at or greater than the first interference threshold in operation 312, the medical device system transmits signals (e.g., signals indicating audio data, auditory stimulation data, visual data, tactile data, olfactory data, taste data, sensory data, electrical stimulation data for various types of electrical stimulation devices (such as pacemakers, defibrillators, seizure devices, sleep apnea devices, electroporation devices), other types of data, software, control codes, etc.) between the external and implantable components concurrently through both the main link and the backup link in operation 313. Otherwise, the medical device system proceeds to operation 303.

In operation 314, the medical device system continues to monitor interference in the signals transmitted through the main link by comparing the interference in the signals transmitted through the main link to the second interference threshold that is greater than the first interference threshold. The first and second interference thresholds can be, for example, thresholds indicative to two different bit error rates. The second interference threshold can be set at the minimum level at which the FEC cannot correct errors in the main link any longer. Alternatively, the second interference threshold can be set just below the minimum level at which the FEC cannot correct errors in the main link in order to prevent audio drop-outs in the main link. If the medical device system determines that the interference in the signals transmitted through the main link is at or greater than the second interference threshold in operation 315, then the medical device system transmits signals (e.g., signals indicating audio data, auditory stimulation data, visual data, tactile data, olfactory data, taste data, sensory data, electrical stimulation data for other types of electrical stimulation devices (such as pacemakers, defibrillators, seizure devices, sleep apnea devices, electroporation devices), other types of data, software, control codes, etc.) between the external and implantable components exclusively through the backup link in operation 307. The medical device system then performs operation 308 to attempt to reestablish communication through the main link, as described above. If the interference in the main link is not at or greater than the second interference threshold in operation 315, the medical device system returns to operation 313.

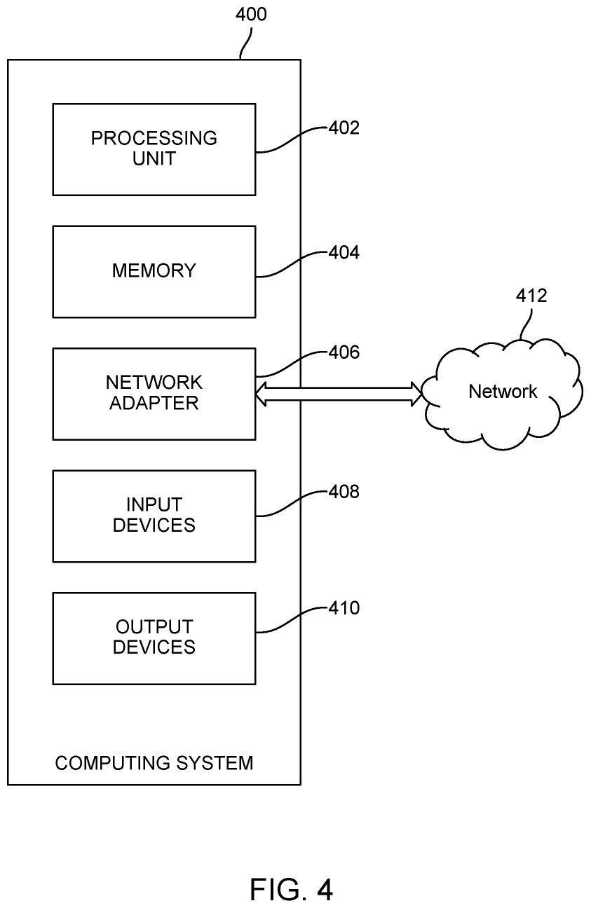

FIG. 4 illustrates an example of a suitable computing system 400 that can perform any of the operations or functions disclosed herein. For example, computing system 400 can be used to perform any one or more of the operations disclosed herein with respect to FIGS. 1-3B. Computing system 400 can be in one or both of the external component 242 and/or the implantable component 244, or external to the medical device system 200. Computing systems, environments, or configurations that can be suitable for use with examples disclosed herein include, but are not limited to, personal computers, server computers, hand-held devices, laptop devices, multiprocessor systems, microprocessor-based systems, programmable consumer electronics (e.g., smart phones), network computers, minicomputers, mainframe computers, tablets, distributed computing environments that include any of the above systems or devices, and the like. The computing system 400 can be a single virtual or physical device operating in a networked environment over communication links to one or more remote devices. The remote device can be an auditory prosthesis (e.g., the auditory prosthesis 100), an ultrasound device, a pressure sensor, a personal computer, a server, a router, a network personal computer, a peer device or other common network node.

Computing system 400 includes at least one processing unit 402 and memory 404. The processing unit 402 includes one or more hardware or software processors (e.g., Central Processing Units) that can obtain and execute instructions. The processing unit 402 can communicate with and control the performance of other components of the computing system 400. The memory 404 is one or more software-based or hardware-based computer-readable storage media operable to store information accessible by the processing unit 402.

The memory 404 can store instructions executable by the processing unit 402 to implement applications (software) or cause performance of any of the functions or operations disclosed herein, as well as store other data. The memory 404 can be volatile memory (e.g., random access memory or RAM), non-volatile memory (e.g., read-only memory or ROM), or combinations thereof. The memory 404 can also include one or more removable or non-removable storage devices. The memory 404 can include transitory memory and/or non-transitory computer-readable storage media. Non-transitory computer-readable storage media is tangible computer-readable storage media that stores data for access at a later time, as opposed to media that only transmits propagating electrical signals, such as wires. In examples, the memory 404 can include non-transitory computer-readable storage media, such as RAM, ROM, EEPROM (Electronically-Erasable Programmable Read-Only Memory), flash memory, optical disc storage, magnetic storage, solid state storage, or any other memory media usable to store information for later access. In examples, the memory 404 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 404 can include wired media such as a wired network or direct-wired connection, and wireless media such as acoustic, radio-frequency, infrared and other wireless media or combinations thereof.

In the illustrated example, the system 400 further includes a network adapter 406, one or more input devices 408, and one or more output devices 410. The system 400 can include other components, such as a system bus, component interfaces, a graphics system, a power source (e.g., a battery), among other components.

The network adapter 406 is a component of the computing system 400 that provides network access to network 412. The network adapter 406 can provide wired or wireless network access and can support one or more of a variety of communication technologies and protocols, such as ETHERNET, cellular, BLUETOOTH, near-field communication, and RF (radio frequency), among others. The network adapter 406 can include one or more antennas and associated components configured for wireless communication according to one or more wireless communication technologies and protocols.

The one or more input devices 408 are devices over which the computing system 400 receives input from a user. The one or more input devices 408 can include physically-actuatable user-interface elements (e.g., buttons, switches, or dials), touch screens, keyboards, mice, pens, and voice input devices, among others input devices.

The one or more output devices 410 are devices by which the computing system 400 is able to provide output to a user. The output devices 410 can include displays, speakers, and printers, among other output devices.

Any embodiment or any feature disclosed herein can be combined with any one or more other embodiments and/or other features disclosed herein, unless explicitly indicated otherwise. Any embodiment or any feature disclosed herein can be explicitly excluded from use with any one or more other embodiments and/or other features disclosed herein, unless explicitly indicated otherwise. It is noted that any method detailed herein also corresponds to a disclosure of a device and/or system configured to execute one or more or all of the method actions associated with the device and/or system as detailed herein. It is further noted that any disclosure of a device and/or system detailed herein corresponds to a method of making and/or using that device and/or system, including a method of using that device according to the functionality detailed herein. The methods can be stored as instructions on non-transitory computer-readable storage media.

The foregoing description of the exemplary embodiments of the present invention has been presented for the purpose of illustration. The foregoing description is not intended to be exhaustive or to limit the present invention to the examples disclosed herein. In some instances, features of the present invention can be employed without a corresponding use of other features as set forth. Many modifications, substitutions, and variations are possible in light of the above teachings, without departing from the scope of the present invention.

1. A medical device system comprising:
a first component, wherein the medical device system switches communication between the first component and a second component of the medical device system from a first communication link to a second communication link in response to an error in the first communication link.

2. The medical device system of claim 1, wherein the first component is an implantable component and the second component is an external component.

3. The medical device system of claim 1, wherein the medical device system attempts to reestablish communication through the first communication link when communication between the first and the second components occurs through the second communication link.

4. The medical device system of claim 1, wherein the medical device system switches communication between the first and the second components from the second communication link to the first communication link in response to detecting interference in the first communication link below a threshold.

5. The medical device system of claim 1, wherein the medical device system transmits communication between the first component and the second component through both the first communication link and through the second communication link in response to an error rate in the first communication link increasing above a first threshold.

6. The medical device system of claim 5, wherein the medical device system transmits data between the first and the second components only through the second communication link in response to the error rate in the first communication link increasing above a second threshold that is greater than the first threshold.

7. The medical device system of claim 1, wherein the medical device system is a cochlear implant system, wherein the cochlear implant system transmits first audio data through the first communication link from the second component to the first component in a first mode, wherein the first component comprises electrodes and a stimulator unit that stimulates the electrodes in response to the first audio data in the first mode, wherein the cochlear implant system transmits second audio data through the second communication link from the second component to the first component in a second mode in response to interference in the first communication link, and wherein the stimulator unit stimulates the electrodes in response to the second audio data in the second mode.

8. An implant system comprising:
an external component, wherein the implant system switches communication between the external component and an implantable component of the implant system from a first communication link to a second communication link in response to interference in the first communication link.

9. The implant system of claim 8, wherein the second communication link operates in a low power standby mode while the first communication link transmits communication between the external and the implantable components.

10. The implant system of claim 9, wherein the implant system causes communications at intervals between the external and the implantable components through the second communication link in the low power standby mode that allow the second communication link to be quickly activated in response to the interference in the first communication link.

11. The implant system of claim 8, wherein the implant system transmits communication between the implantable and the external components through both of the first and the second communication links in response to the interference in the first communication link increasing above a first threshold, and
wherein the implant system transmits data signals between the external and the implantable components only through the second communication link in response to the interference in the first communication link increasing above a second threshold that is greater than the first threshold.

12. The implant system of claim 8, wherein the implant system monitors the interference in the first communication link when communication between the external and the implantable components occurs through the second communication link to determine when to reestablish communication between the external and the implantable components through the first communication link.

13. A method comprising:
communicating between external and implantable components of an implant system through a first signal transmission link; and
communicating between the external and the implantable components through a second signal transmission link in response to detecting interference in the first signal transmission link.

14. The method of claim 13 further comprising:
monitoring the interference in the first signal transmission link while communication between the external and the implantable components occurs through the second signal transmission link; and
switching communication between the external and the implantable components from the second signal transmission link to the first signal transmission link in response to detecting the interference in the first signal transmission link is less than a threshold.

15. The method of claim 13 further comprising:
comparing the interference in signals transmitted between the external and the implantable components through the first signal transmission link to a predefined value; and
switching communication between the external and the implantable components from the first signal transmission link to the second signal transmission link in response to detecting that the interference is greater than the predefined value.

16. The method of claim 13 further comprising:
operating the second signal transmission link in a low power standby mode while communication between the external and the implantable components occurs through the first signal transmission link.

17. The method of claim 16, wherein operating the second signal transmission link in the low power standby mode further comprises transmitting communication for at least one function of an automated handshaking process through the second signal transmission link at intervals that are spaced apart in time.

18. The method of claim 13, wherein the first signal transmission link is a magnetic induction link, and wherein the second signal transmission link is a radio frequency link.

19. (canceled)

20. (canceled)

21. (canceled)

22. (canceled)

23. (canceled)

24. (canceled)

25. (canceled)

26. The method of claim 13 further comprising:
detecting when an error rate of bits transmitted through the first signal transmission link reaches a threshold,
wherein communicating between the external and the implantable components through the second signal transmission link further comprises causing signals to be transmitted through the second signal transmission link in response to the error rate reaching the threshold.

27. The method of claim 26 further comprising:
switching transmission of data between a first processing module in the external component and a second processing module in the implantable component from the first signal transmission link to the second signal transmission link in response to the error rate reaching the threshold.
